(19) United States
(12) Patent Application Publication
(10) Pub. No.: US 2010/0041922 A1
(43) Pub. Date: Feb. 18, 2010

(54) METHOD FOR THE ENANTIOSELECTIVE PRODUCTION OF OPTICALLY ACTIVE 4-HYDROXY-2,6,6-TRIMETHYL-CYCLOHEX-2-ENONE DERIVATIVES

(75) Inventors: Rainer Stürmer, Rodersheim-Gronau (DE); Hansgeorg Ernst, Speyer (DE)

Correspondence Address: CONNOLLY BOVE LODGE & HUTZ, LLP, P O BOX 2207, WILMINGTON, DE, 19899, US

(73) Assignee: BASF SE, Ludqigshafen (DE)

(21) Appl. No.: 12/593,466

(22) PCT Filed: Feb. 22, 2008
(86) PCT No.: PCT/EP2008/052202

§ 371 Date: Sep. 28, 2009

(30) Foreign Application Priority Data
Mar. 28, 2007 (EP) 07105089.2

(51) Int. Cl. C07C 45/61

(52) U.S. Cl. 568/343

(57) ABSTRACT

The present invention relates to a process for enantioselectively preparing optically active 4-hydroxy-2,6,6-trimethylcyclohex-2-en-1-one derivatives of the formulae (I) or (Ia) and to a process for preparing (3S,3′S)-astaxanthin of the formula (III), comprising the process for preparing the compound of the formula (I).

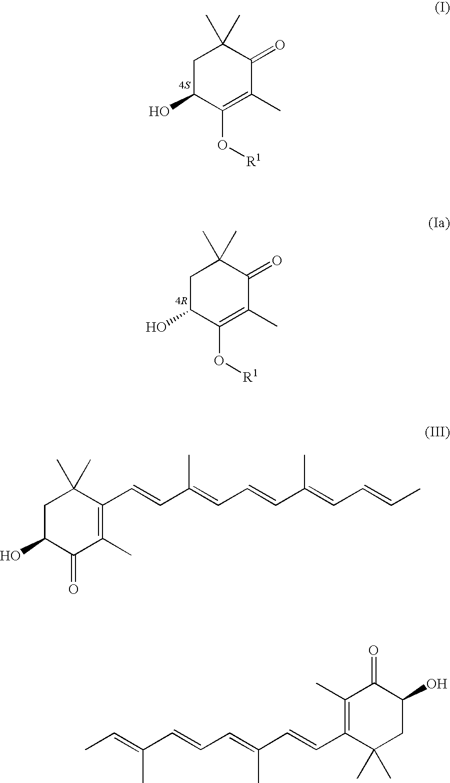

The present invention relates to a process for enantioselectively preparing optically active 4-hydroxy-2,6,6-trimethylcyclohex-2-en-1-one derivatives of the formulae (I) or (Ia) and to a process for preparing (3S,3′S)-astaxanthin of the formula (III), comprising the process for preparing the compound of the formula (I).

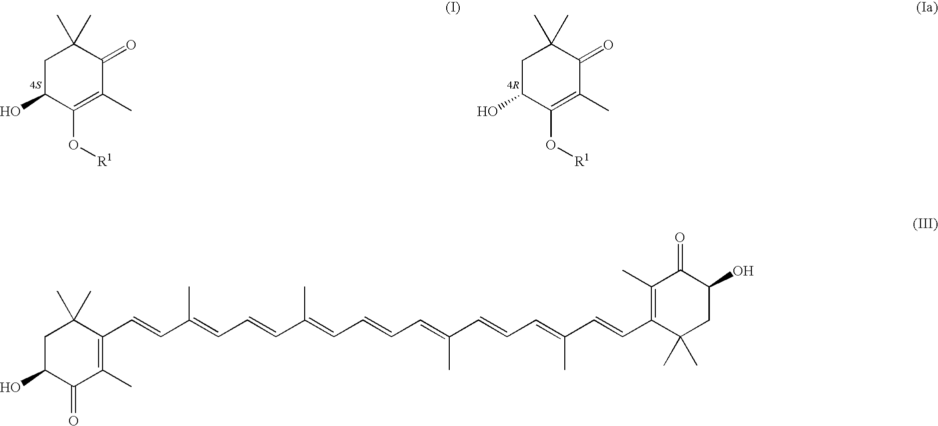

Owing to its two chiral centers in the 3 and 3′ position, astaxanthin (3,3′-dihydroxy-β,β′-carotene-4,4′-dione) may be present in the form of the following configuration isomers: (3S,3′S), (3R,3′R), (3S,3′R) and (3R,3′S). The latter two configuration isomers are identical and constitute a meso form (Carotenoids Handbook, 2004, Main List Nr. 405).

All three forms are found in natural sources (Carotenoids Handbook, 2004, Main List No. 404, 405, 406). The chemical total synthesis proceeding from racemic precursors leads to a 1:2:1 mixture of (3S,3′S)-, meso- and (3R,3′R)-astaxanthin (The EFSA Journal, 2005, 291, 12, Deutsche Lebensmittel-Rundschau, 2004, 100, 437, Helvetica Chimica Acta, 1981, 64, 2436).

However, the (3S,3′S) configuration isomer is of particular significance. It is biosynthesized in enantiomerically pure form by green algae (Haematococcus pluvialis) (J. Applied Phycology, 1992, 4, 165; Phytochemistry, 1981, 20, 2561).

(S,S)-Astaxanthin from green algae is used as a food supplement with positive effects on human health (J. Nat. Prod., 2006, 69, 443). Furthermore, it is suitable for completely blocking the disadvantageous prooxidative effects of rofecoxib (Vioxx) (J. Cardiovasc. Pharmacol., 2006, 47 Suppl 1, p. 7).

In view of the low concentration of (S,S)-astaxanthin in green algae (J. Agric. Food Chem., 1998, 46, 3371), the availability of this active ingredient is, however, very limited. In addition, the active ingredient in the algae is present in a mixture of mono- and di-fatty acid esters and free astaxanthin, which causes a considerable level of complexity for the isolation and purification (see, inter alia, Phytochemistry, 20, 11, 2561 (1981); J. Applied Phycology, 4, 2, 165 (1992)). In order to be able to provide (S,S)-astaxanthin in a larger amount and high purity, chemical total synthesis is the technology of choice.

Various syntheses of (S,S)-astaxanthin have been described in the literature. One strategy consists in the splitting of racemic precursors into the optical antipodes, using diastereomeric salts (Helvetica Chimica Acta, 1981, 64, 2447) or diastereomeric esters (Helvetica Chimica Acta, 1981, 64, 2419). There have also been reports of microbial optical resolution of racemic precursors. What is particularly disadvantageous about these processes is that the enantiomer that would lead to (R,R)-astaxanthin is unusable or can be recycled only with very great difficulty.

Another synthesis strategy consists in arriving at enantiomerically pure synthesis units via microbial or enzymatic processes (Helvetica Chimica Acta, 1978, 61, 2609, Helvetica Chimica Acta, 1981, 64, 2405). Since these units have too low an oxidation state, they have to be converted to (S,S)-astaxanthin precursors in multistage syntheses.

Firstly, WO 2006/039685 describes, in scheme II, a two-stage enantioselective hydrogenation of ketoisophorone to an enantiomerically pure C9-diol, from which, after reoxidation of one hydroxyl group, based on the process described in Helv. Chim. Acta, 1978, 61, 2609, (S,S)-astaxanthin precursors are arrived at in a multistage synthesis. Moreover, WO 2006/039685 describes an enantioselective catalytic transfer hydrogenation of a C9-enol ether of the formula (II-a) to the corresponding enantiomerically pure alcohol of the formula (I-b).

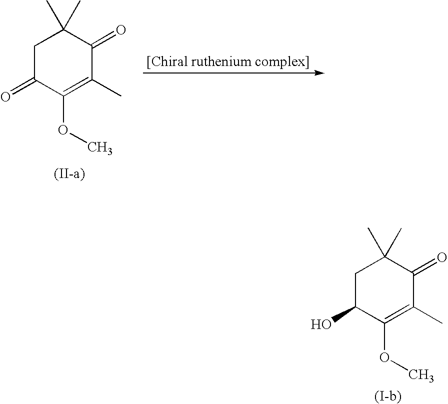

The hydrogenation catalysts described are metals with chiral ligands, preferably ruthenium catalysts with optically active amines as ligands. What is disadvantageous about this process is that an oxygen-protected derivative of an industrial intermediate of the formula (II—OH) is used

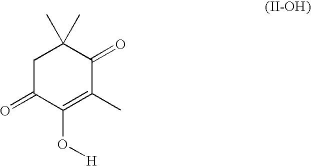

which entails additional synthetic complexity.

It was an object of the present invention, proceeding from industrially available starting materials, to develop a simplified and economically efficient process for preparing an optically active intermediate, as far as possible in enantiomerically pure form, for the synthesis of (S,S′)-astaxanthin or (R,R′)-astaxanthin, which can be integrated without any problem into the existing industrial total syntheses of “racemic” astaxanthin (Carotenoids Vol. 2, 1996, 259; Pure and Applied Chemistry, 2002, 74, 2213).

This object is achieved by a process for enantioselectively preparing optically active 4-hydroxy-2,6,6-trimethylcyclohex-2-en-1-one derivatives of the formulae (I) or (Ia)

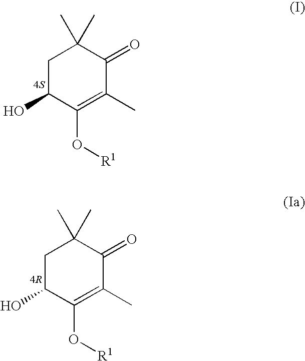

by reacting a trimethylcyclohex-2-ene-1,4-dione derivative of the formula (II),

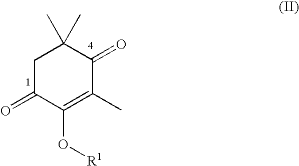

in which, in the formulae (I), (Ia) and (II),

R¹ is an alkali metal M¹ or an alkaline earth metal fragment M²_{1/2} or (M²)⁺X⁻, where M¹ is Li, Na, K, Rb or Cs and M² is Mg, Ca, Sr or Ba and X⁻ is a singly charged anion, in the presence of a reducing agent RA and of a chiral transition metal catalyst to give a compound of the formula (I) or (Ia), the carbonyl group in position 1 of the compound of the formula (II) being hydrogenated in the presence of the chiral transition metal catalyst either preferentially to the secondary (4S)-alcohol of the formula (I) or preferentially to the secondary (4R)-alcohol of the formula (Ia), and the oxidized reducing agent RA, if appropriate, being removed at least partially from the reaction equilibrium.

In the process according to the invention, compounds of the formula (II) are used and compounds of the formulae (I) or (Ia) are prepared in which R¹ is an alkali metal M¹ or an alkaline earth metal fragment M²_{1/2} or (M²)⁺X⁻, where M¹ is Li, Na, K, Rb or Cs, preferably Na or K, especially Na, and M² is Mg, Ca, Sr or Ba, especially Mg, and X⁻ is a singly charged anion, for example halide, acetate or dihydrogenphosphate. R¹ is preferably Na or K, especially Na.

In the process according to the invention, the compound of the formula (II) is converted in the presence of a chiral transition metal catalyst to a compound of the formula (I) or (Ia).

The chiral transition metal catalyst which is suitable for the reduction of the keto group on carbon atom 4 to the secondary alcohol preferably comprises a transition metal atom and at least one optically active chiral ligand. Useful transition metal atoms are in principle all transition metals, for example Ti, Zr, Hf, V, Nb, Ta, Cr, Mo, W, Mn, Re, Fe, Ru, Os, Co, Rh, Ir, Ni, Pd, Pt, Cu, Ag or Au, which can form a suitable chiral transition metal catalyst.

In the process according to the invention, particular preference is given to using a chiral transition metal catalyst which comprises a transition metal atom and at least one optically active chiral ligand, where the transition metal atom is ruthenium. Preferred chiral ruthenium catalysts can be obtained, for example, by reacting a suitable ruthenium compound, for example [RuX₂(η⁶-Ar)]₂, with a suitable chiral ligand, where X is a halogen atom such as fluorine, chlorine, bromine or iodine, and Ar is benzene or a substituted benzene derivative, especially a benzene derivative substituted by C₁-C₄-alkyl radicals.

In the chiral ruthenium catalyst, the optically active chiral ligand is preferably an optically active amine or an optically active amino acid. Examples of optically active amines which can be reacted with a suitable ruthenium compound, especially [RuX₂(η⁶-Ar)]₂, to give the catalytically active complex are, for example, H₂N—CHPh-CHPh-OH, H₂N—CHMe-CHPh-OH, MeHN—CHMe-CHPh-OH or TsNH—CHPh-CHPh-NH₂, especially (1S,2S)—N-p-toluenesulfonyl-1,2-diphenylethylenediamine or (1R,2R)—N-p-toluenesulfonyl-1,2-diphenylethylenediamine.

Particular preference is given to a chiral ruthenium catalyst in which the optically active chiral ligand is obtainable by single deprotonation of H₂N—CHPh-CHPh-OH, H₂N—CHMe-CHPh-OH, MeHN—CHMe-CHPh-OH or TsNH—CHPh-CHPh-NH₂, especially by single deprotonation of (1S,2S)—N-p-toluenesulfonyl-1,2-diphenylethylenediamine or (1R,2R)—N-p-toluenesulfonyl-1,2-diphenylethylenediamine.

When, for example, (1S,2S)—N-p-toluenesulfonyl-1,2-diphenylethylenediamine is used as the optically active chiral ligand in the process according to the invention, the compound of the formula (I) is obtained in high enantiomeric purity

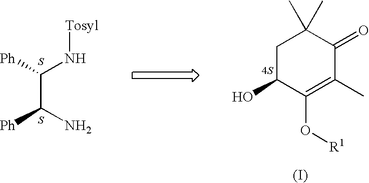

while, in the case of use of (1R,2R)—N-p-toluenesulfonyl-1,2-diphenylethylenediamine, the optically active chiral ligand obtained is the compound of the formula (Ia).

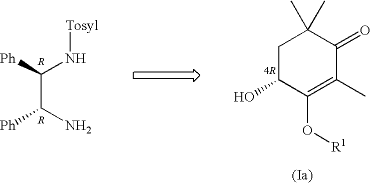

In the process according to the invention, the reducing agents RA used may in principle be inorganic or organic compounds, for example hydrogen or alcohols. In the process according to the invention, the reducing agent RA used is preferably an organic compound which comprises at least one primary or secondary alcohol function CH(OH), for example isopropanol, 2-butanol, 2-pentanol, 2-hexanol or 3-hexanol, especially isopropanol. The oxidized reducing agent RA formed in the process according to the invention, for example acetone in the case of use of isopropanol as the reducing agent RA, can be removed at least partially from the reaction medium or from the reaction equilibrium. When the reducing agent RA is a secondary alcohol, this is frequently also referred to as a sacrificial alcohol, and the correspondingly formed oxidation product as a sacrificial ketone.

The process according to the invention is performed typically in the liquid phase, i.e. In at least one solvent or solvent mixture. The liquid phase preferably comprises at least one organic solvent, in which case the liquid phase typically consists of organic solvents to an extent of more than 50% by volume. As an inorganic solvent, the liquid phase may comprise especially water. According to the composition of the liquid phase of different solvents, the liquid phase may be a monophasic, biphasic or else multiphasic system. The process according to the invention is preferably performed in a monophasic system, in which case the solvent used is especially mixtures of a secondary alcohol, especially isopropanol, and water.

In the process according to the invention, the oxidation product formed can be removed at least partially from the reaction medium or from the reaction equilibrium. In the case of secondary alcohols such as isopropanol as the reducing agent (RA), the removal of the so-called sacrificial ketones formed, acetone in the case of isopropanol as the sacrificial alcohol, can be undertaken in various ways, for example by means of selective membranes or by means of extraction or distillation processes.

The process according to the invention is performed advantageously at a temperature between 0° C. and 150° C., preferably between 10° C. and 85° C., more preferably between 15° C. and 75° C.

In the process according to the invention, optically active compounds are understood to mean enantiomers which exhibit enantiomeric enrichment. In the process according to the invention, preference is given to achieving enantiomeric purities of at least 70% ee, preferably of min. 80% ee, more preferably of min. 90% ee, most preferably min. 98% ee.

The compounds of the formulae (I) or (Ia) prepared in the process according to the invention can be converted by acidification to the corresponding diol, for example (4S)-3,4-dihydroxy-2,6,6-trimethylcyclohex-2-enone, which can be obtained by known methods, for example extraction or precipitation. One method of isolating the diol of the formulae (I—OH) or (Ia-OH) obtainable after acidification

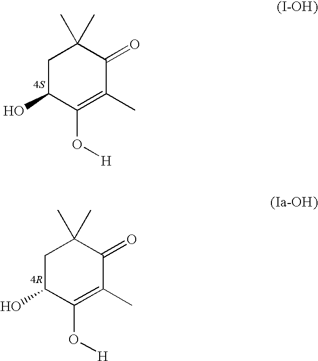

can in principle be effected as described in Helv. Chim. Acta 64, 2436, 1981. After removal of organic solvents or after dilution with water, the product solution can first be adjusted to a pH of from 1 to 3, preferably pH 1. The acidification is performed preferably with mineral acids, for instance hydrochloric acid or sulfuric acid, more preferably with sulfuric acid. The product frequently precipitates out and can be removed, or the acidic product solution is extracted repeatedly with an organic water-immiscible solvent. Suitable solvents here are chlorinated hydrocarbons, especially methylene chloride, ethers, for instance MTBE or diisopropyl ether, and ethyl acetate. This extraction can be performed batchwise or continuously. The extraction of the product can be supported by concentration of the aqueous phase before the acidification or by “salting-out”; however, these operations are not essential for the removal of the product from the reaction solution.

In the process according to the invention, the product of the formula (I) or (Ia) can be prepared in yields of from 60 to more than 95%, preferably from 80 to more than 95%, based on the substrate of the formula (II) used in the reaction (for example where R²═Na) and, after workup, can be isolated as the diol of the formula (I—OH) or (Ia-OH). The diol can be obtained in an enantiomeric purity of more than 98% ee. It can, if desired, be purified by crystallization according to Helv. Chim. Acta 64, 2436, 1981, but is preferably used without further purifying operations in the further synthesis of S,S-astaxanthin according to Helv. Chim. Acta 64, 2447, 1981.

The process according to the invention can be performed batchwise, semibatchwise or continuously.

The invention further provides a process for preparing (3S,3′S)-astaxanthin of the formula (III)

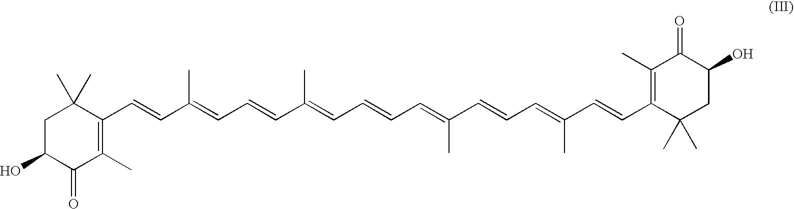

by, in one reaction step of the overall synthesis of (3S,3′S)-astaxanthin, preparing the compound of the formula (I) prepared in the above-described process according to the invention

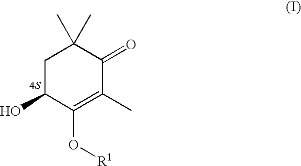

in the process according to the invention.

(3R,3′R)-Astaxanthin can be prepared analogously using a compound of the formula (Ia).

Both the synthesis steps for preparing the starting compounds of the formula (II) and the synthesis steps for converting the enantiomerically pure compound of the formula (I) or of the formula (Ia) via several stages to (3S,3′S)-astaxanthin of the formula (III) or (3R,3′R)-astaxanthin are known in principle from the literature. The conversion of the optically pure compound of the formula (I) or of the formula (Ia) obtained by enantioselective reduction of the compound of the formula (II) in which R² is preferably Na to (3S,3′S)-astaxanthin or (3R,3′R)-astaxanthin is effected without racemization as described on various occasions in the literature (WO 2006/039685; Helv. Chim. Acta, 1981, 64, 2447; ibid., 1981, 64, 2405).

These processes correspond to industrial astaxanthin syntheses (Carotenoids, Vol. 2, 1996, 259; Pure and Appl. Chem., 2002, 74, 2213) and offer a technically and economically advantageous route to (3S,3′S)-astaxanthin.

The advantage of the process according to the invention lies in the simplification of obtaining compounds of the formulae (I) or (Ia) with high enantiomeric purity combined with good yields of these compounds.

The invention is illustrated by the following examples which do not, however, restrict the invention.

### EXAMPLES

### Example 1

### Analysis of the Compounds (I—OH), (Ia-OH) and (II—OH)

The reactant and product concentrations can be determined by means of HPLC. According to the selection of the stationary and mobile phase, it is also possible to determine ee as well as the concentration.

| Stationary | Chiralpak AS-RH, 150 * 4.6 mm, Daicel, heated to 40° C. | | | |
|---|---|---|---|---|
| phase: | | | | |
| Mobile | Eluent A: 10 mM KH₂PO₄ | | | |
| phase: | | | | |
| Eluent B: | CH₃CN | | | |
| | Time [min] | A [%] | B [%] | Flow rate [ml/min] |
| Gradient: | 0 | 90 | 10 | 0.5 |
| | 10 | 90 | 10 | 0.5 |
| | 11 | 60 | 40 | 0.5 |
| | 20 | 60 | 40 | 0.5 |

| | |
|---|---|
| Flow rate: | 0.5 ml/min |
| Detection: | UV detection at 260 nm |
| Retention | |
| times: | |
| (Ia-OH) | (4R)-(+)-3,4-Dihydroxy-2,6,6-trimethylcyclohex-2-enone: |
| | approx. 9.3 min |
| (I-OH) | (4S)-(−)-3,4-Dihydroxy-2,6,6-trimethylcyclohex-2-enone: |
| | approx. 9.8 min |
| (II-OH) | 2-Hydroxy-3,5,5-trimethylcyclohex-2-ene-1,4-dione: |
| | approx. 17.6 min |

Authentic material is used to establish a calibration series, with the aid of which the concentration of unknown samples can be determined and the assignment of the enantiomers is enabled.

### Example 2

### Preparation of sodium (4S)-4-hydroxy-2,6,6-trimethyl-1-oxocyclohex-2-en-3-olate (I—Na)

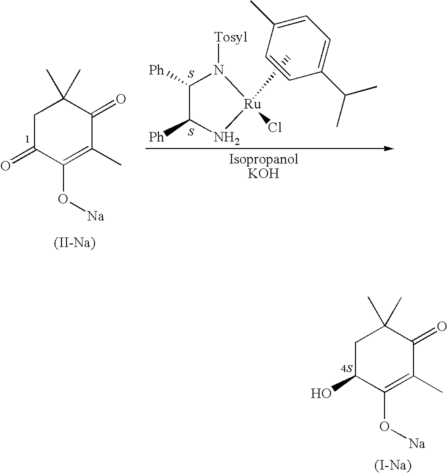

Under a nitrogen atmosphere, 10 ml of isopropanol and 26.8 mg of (1S,2S)-(+)-N-p-toluenesulfonyl-1,2-diphenylethylenediamine and 11.8 mg of dichloro(p-cymene)ruthenium(II) dimer were initially charged. The mixture was heated to 80° C. under nitrogen for 30 min, then cooled to 20° C. and admixed with 2.4 ml of a degassed aqueous solution of sodium 3,5,5-trimethyl-1,4-dioxocyclohex-2-en-2-olate (II—Na) in 2.4 ml of isopropanol. After addition of 1.8 ml of 0.1 M potassium hydroxide solution in isopropanol, the mixture was stirred at 40° C. for 24 h. After acidification, it was found by means of HPLC analysis that the compound (II—Na) had been converted quantitatively and the chiral alcohol (I—Na) had an enantiomeric purity of greater than 99% ee.

### Example 3

### Preparation of sodium (4R)-4-hydroxy-2,6,6-trimethyl-1-oxocyclohex-2-en-3-olate (Ia-Na)

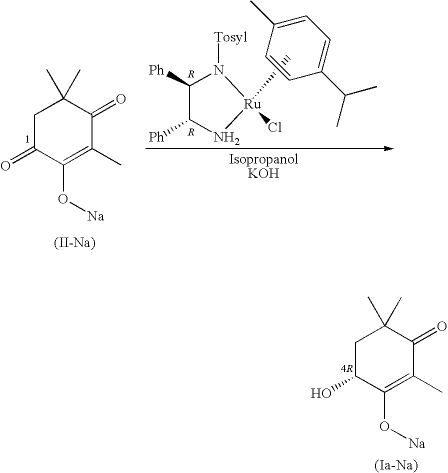

Analogously to Example 2, a chiral catalyst was prepared by reacting 26.8 mg of (1R,2R)-(−)-N-p-toluenesulfonyl-1,2-diphenylethylenediamine with 11.8 mg of dichloro(p-cymene)ruthenium(II) dimer, and, according to Example 2, used to reduce the compound (II—Na) to the compound (Ia-OH), and a quantitative conversion was achieved and the product has an enantioselectivity greater than 99% ee.

### Example 4

### Preparation of sodium (4S)-4-hydroxy-2,6,6-trimethyl-1-oxocyclohex-2-en-3-olate (I—Na)

Analogously to Example 2, a tenth of the amount of catalyst (SIC 1000:1) from Example 2 was used to perform the reduction of compound (II—Na). The reaction monitoring by means of HPLC analysis, after 24 h, gave 72% conversion and, after 48 h, full conversion with identical enantiomeric excesses

1-9. (canceled)

10. A process for enantioselectively preparing optically active 4-hydroxy-2,6,6-trimethylcyclohex-2-en-1-one derivatives of the formulae (I) or (Ia) 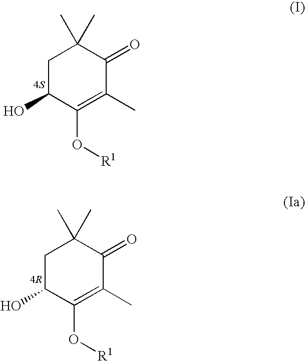
by reacting a trimethylcyclohex-2-ene-1,4-dione derivative of the formula (II), 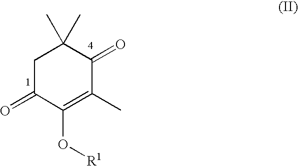
in which, in the formulae (I), (Ia) and (II),
R¹ is an alkali metal M¹ or an alkaline earth metal fragment M²_{1/2} or (M²)⁺X⁻, where M¹ is Li, Na, K, Rb or Cs and M² is Mg, Ca, Sr or Ba and X⁻is a singly charged anion,
in the presence of a reducing agent and of a chiral transition metal catalyst RA to give a compound of the formula (I) or (Ia), the carbonyl group in position 1 of the compound of the formula (II) being hydrogenated in the presence of the chiral transition metal catalyst either preferentially to the secondary (4S)-alcohol of the formula (I) or preferentially to the secondary (4R)-alcohol of the formula (Ia), and the oxidized reducing agent RA, if appropriate, being removed at least partially from the reaction equilibrium.

11. The process according to claim 10, wherein the reducing agent RA used is an organic compound which comprises at least one primary or secondary alcohol function CH(OH), especially isopropanol.

12. The process according to claim 10, wherein R¹ in the formulae (I), (Ia) and (II) is sodium.

13. The process according to claim 10, wherein the chiral transition metal catalyst comprises a transition metal atom and at least one optically active chiral ligand.

14. The process according to claim 13, wherein the transition metal atom is ruthenium.

15. The process according to claim 14, wherein the optically active chiral ligand is an optically active amine or an optically active amino acid.

16. The process according, to claim 14, wherein the optically active chiral ligand is obtainable by simple deprotonation of H₂N—CHPh-CHPh-OH, H₂N—CHMe-CHPh-OH, MeHN—CHMe-CHPh-OH or TsNH—CHPh-CHPh-NH₂.

17. The process according to claim 14, wherein the optically active chiral ligand is obtainable by simple deprotonation of (1S,2S)—N-p-toluenesulfonyl-1,2-diphenylethylenediamine or (1R,2R)—N-p-toluenesulfonyl-1,2-diphenylethylenediamine.

18. A process for preparing (3S,3′S)-astaxanthin, wherein, in one reaction step of the overall synthesis of (3S,3′S)-astaxanthin, the compound of the formula (I) prepared in claim 10 is prepared by the process according to claim 10.
